(19) United States
(12) Patent Application Publication
(10) Pub. No.: US 2005/0063510 A1
(43) Pub. Date: Mar. 24, 2005

(54) Radiotherapy system

(76) Inventors: Christian Hieronimi, Graben (DE); Michael Vogele, Schwabmunchen (DE)

Correspondence Address: THOMAS J. TIGHE & ASSOCIATES, 6265 GREENWICH DRIVE, SUITE 103, SAN DIEGO, CA, 92122, US

(21) Appl. No.: 10/498,930

(22) PCT Filed: Dec. 12, 2002
(86) PCT No.: PCT/EP02/14163

(30) Foreign Application Priority Data
Dec. 12, 2001 (DE) 101 61 152.8

(51) Int. Cl. A61N 5/10

(52) U.S. Cl. 378/65

(57) ABSTRACT

The invention relates to a radiotherapy system for directing a treatment beam onto an isocenter (14) in a patient (13), especially for tumor treatment in radiotherapy. Said radiotherapy system comprises a base (15) on which the patient (13) rest$ and a radiation device, more particularly, a lincar accelerator (1) that generates a treatment beam (12). According to the invention, the direction of the treatment beam (12) can be regulated by means of a hexapode (3, 4, 5, 6, 7, 8, 9, 10).

The invention relates to a radiotherapy system according to the features of the preamble of claim 1.

Known radiotherapy systems consist at least of one base on which the patient rests, namely the so-called patient's berth, and a radiation apparatus, in particular a so-called linear accelerator. The linear accelerator is usually fastened to a frame, the so-called gantry. The gantry is usually provided with a movable configuration, i.e. it is rotatable about the patient situated on the berth. The radiation field produced in the linear accelerator is focused in a focusing instrument, namely the so-called collimator, and optionally shaped, i.e. the shape of the radiation field is adjusted to the shape of the tumor, thus enabling a purposeful irradiation.

One problem encountered in radiotherapy is to position the tumor and thus the patient relative to the radiation source in such way that the ray or the radiation field hits the tumor as precisely as possible and avoids adjacent tissue. Principally there are two possibilities which can also be combined. On the one hand, the radiation source can remain stationary and the patient and thus the tumor can be moved relative to the same. On the other hand, the patient can be fixed and the radiation source can be moved. In order to change the position of the patient, various systems are known which are all based on the fact that the patient who is fastened to the berth is moved in such a way that the position of the berth is adjusted.

DE 197 28 788 describes a method for the positioning of patients relative to the treatment device. The patient's actual position is determined here by means of CCD cameras and image processing and morphing and compared with a previously determined desired position. Thereupon the servomotors of the berth are triggered which bring the patient back to the set position. This triggering is performed in the second or tenths of second cycle in order to also allow responding to the breathing action of the patient.

A method is further known from DE 198 05 917 with which the position of patients can be recognized during radiotherapy and the patient can be positioned accordingly. For this purpose the surface structure of the patient's body is detected with at least two sensors and compared with a set image, as a result of which deviations of the current position of the patient from the desired position can be recognized. Thereupon it is possible to optionally perform a correction of the positional deviation.

In the adjustment of the radiation source it is also known that the same can occur by rotation of the gantry. U.S. Pat. No. 6,052,436 further shows an apparatus for radiotherapy in which two guide rails are fixed over the patient on which a linear accelerator with attached collimator can be moved. As a result of slots in the guide rails, the plates of the collimator are moved in such a way that the radiation window changes following the motion of the linear accelerator, such that the shape of the radiation window is adjusted to the shape of the tumor.

Despite the known solutions, the problem remains that the positioning of the patient or, in other words, the isocenter of the tumor relative to the radiation source is still relatively imprecise. Moreover, the known radiation systems come with the disadvantage that the radiation source is adjustable only within limits relative to the patient, as a result of which the irradiation from unusual angles is made more difficult or complex apparatuses are necessary.

It is the object of the present invention to provide a radiation therapy system which avoids the aforementioned disadvantages. A system is to be provided with which the radiation source can be set relative to the patient in the quickest and most precise manner in order to achieve an optimal treatment of the tumor.

This object is achieved by a radiotherapy system pursuant to claim 1. Advantageous embodiments are the subject matter of the subclaims.

The radiation therapy system in accordance with the invention consists at least of a base on which the patient rests and a radiation apparatus, especially a linear accelerator, which generates a treatment beam. The term “treatment beam” shall designate all types of radiation produced by the linear accelerator, i.e. both photon as well as electron rays. Moreover, the term shall not only comprise point-like ray beams, but also so-called radiation fields. It is provided for according to the invention that the direction of the treatment beam is adjustable by means of at least one hexapode. The term “hexapode” designates an apparatus which works according to the so-called Stewart principle (D. Stewart, “A Platform With Six Degrees of Freedom”), UK Institution of Mechanical Engineers Proceedings, 1965-66, Vol. 180, Pt 1, No 15). A hexapode comprises six struts or stays, especially hydraulic cylinders or electric spindles, which each extend between an upper and a lower platform. One of the two platforms is fixed or stationary, whereas the other is moved by change in the length of the struts, stays or spindles. The hexapode allows a combined translational and rotary movement along or about the six coordinates (X, Y, Z, theta-X, theta-Y, theta-Z). As a result, a hexapode has six degrees of freedom. The use of a hexapode for defining the direction of the treatment beam therefore allows its rapid and precise alignment. This means in practical operation that by rotating the gantry there is a rough alignment and the fine adjustment can then occur especially by means of the hexapode, such that the treatment beam is aligned with the hexapode. This allows an especially rapid and precise adjustment. The use of the hexapode for aligning the treatment beam further allows that there is low need for space in comparison with other adjusting possibilities (such as so-called compound tables). The hexapod further has a relatively small overall size.

It is preferably provided that the hexapod is attached between linear accelerator and collimator, and especially that the same is inserted with a ring disk. Furthermore, it is preferably provided that at least one sensor is provided on the hexapode and/or the linear accelerator and/or the collimator with which the position of the patient can be detected.

Preferably, two sensors are provided. It can also be provided that only one sensor is provided on the hexapod or linear accelerator or collimator and the other at any other desirable point in the treatment room. This ensures a precise determination of the position of the patient because at least two images are produced and can be compared with each other.

An especially preferable embodiment of the invention provides that the hexapod can be controlled in such a way that the treatment beam can be guided according to the shape of the tumor. Such a control can provide for example that by means of methods which produce a three-dimensional picture such as computer tomography (CT), it is possible to detect the shape of the tumor and the position of the tumor in the patient. On the basis of such data, the treatment beam is thus aligned and moved by means of the hexapod and the beam guide elements as set by the same that the treatment beam follows the shape of the tumor. It is ensured in this way that the tumor is irradiated fully and it is prevented at the same time that adjacent tissue is affected by the irradiation. Moreover, the guidance of the treatment beam along the shape of the tumor ensures that it is possible to work with the lowest possible dose because any factors of uncertainty concerning the tumor size for example can be excluded and its purposeful irradiation is ensured.

The invention is explained and described in closer detail by reference to the enclosed drawing, wherein:

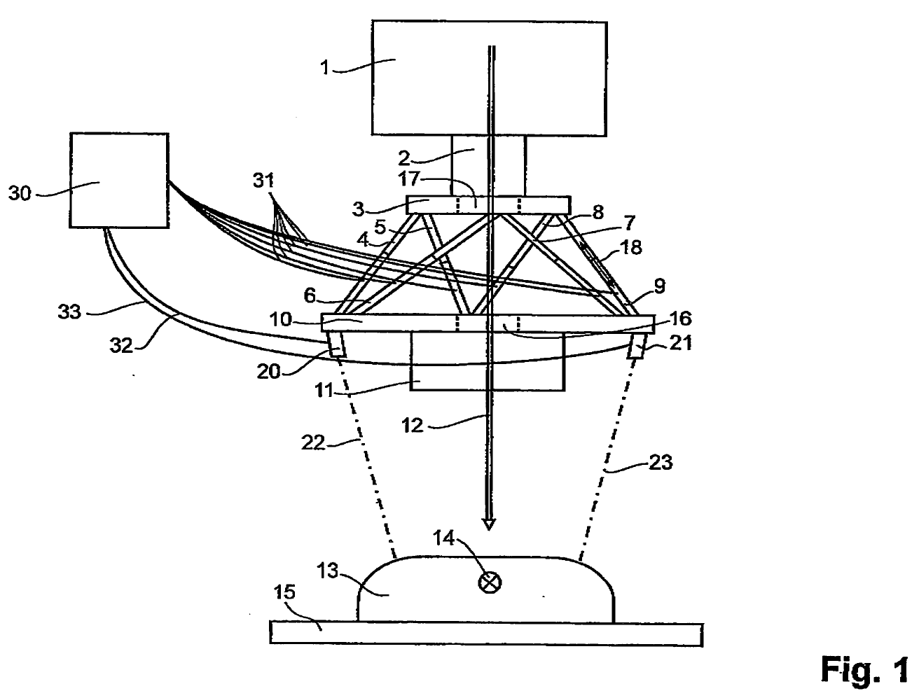

FIG. 1 shows a radiotherapy system in accordance with the invention in a schematic representation.

As is shown in the (only) FIG. 1, the radiotherapy system in accordance with the invention comprises a linear accelerator 1. The linear accelerator 1 can assume any desirable shape. It can be arranged as a device standing on the floor, but also as a device mounted on the ceiling. The linear accelerator 1 will usually be fastened to a frame, the so-called gantry. The radiation required for the treatment will be produced in the known manner in the linear accelerator. The treatment beam 12, which is indicated by a respective arrow, thus passes the head 2 of the linear accelerator 1. It is provided for in accordance with the invention that a hexapode is provided between the head 2 of the linear accelerator 1 and a collimator 11. The hexapode comprises two platforms 3 and 10, with the platform 3 being fastened to the linear accelerator 1, preferably to its head 2, and the movable platform 10 is fastened to collimator 11 for its adjustment. Instead of the collimator 11 it is possible to provide any other desirable focusing or beam guide element, depending on the desired application.

The platforms 3 and 10 of the hexapode are provided with a ring-like configuration and therefore comprise pass-through openings 16 and 17 through which the treatment beam 12 passes. It is preferably provided that the platform 3 is fixedly connected with the linear accelerator 1 or its head 2, and thus forms the platform of the hexapode which is fixed in its position. Platform 10 is adjustable by changing the length of the struts 4, 5, 6, 7, 8 and 9, with the term “struts” also relating to similarly acting stays or spindles or general translational drives. The struts 4, 5, 6, 7, 8 and/or 9 are longitudinally adjustable along their longitudinal axis, as is indicated by arrow 18. By changing the length of at least one strut 4, 5, 6, 7, 8 and/or 9 the adjustable platform 10 is thus changed in its position and thus the collimator 11 as a beam guide element is co-moved accordingly. This again changes the irradiation angle of the treatment beam 12. The treatment beam 12 can thus be aligned in such a way that as precisely as possible it meets the isocenter 14 in the patient who is strapped to a base 15. Such an isocenter 14 is understood as being a tumor for example which is to be treated by means of radiotherapy.

Preferably, a sensor system is provided with which the position of patient 13 can be fixed to the base 15. Sensors 20 and 21 can be provided on platform 10 for example. Scanning systems can be used as sensors 20 and 21 which continuously scan the body and thus the position of patient 13 or which scan the surface shape of patient 13. The sensors 20 and 21 are oriented towards the patient 13, as is indicated by the dot-dash lines 22 and 23. The sensors 20 and 21 are thus used to detect the position of patient 13 on the base 15 and thus to check continually whether the isocenter 14 and the treatment beam 12 are aligned optimally with respect to each other, meaning whether the treatment beam 12 meets the isocenter 14 precisely. If deviations are determined then it is provided that the aforementioned hexapode is controlled in such a way that the treatment beam 12 is readjusted and the same meets the isocenter 14 again precisely.

As an alternative it could also be provided that in the case of a deviation from the actual position of patient 13 from the scheduled position, the radiation system is cut off in order to prevent any damage to ambient tissue. FIG. 1 schematically shows a control unit 30 which is connected with the hexapode via the one signal output 31 each. The control unit 30 can further comprise different inputs, e.g. the inputs 32 and 33 of sensors 20 and 21. Moreover, the control unit 30 can also comprise signal inputs of imaging devices such as a CT. It is provided that the control unit 30 triggers each strut 4, 5, 6, 7, 8, 9 of the hexapode permanently and separately in order to achieve the most precise possible alignment of the treatment beam 12 in all six degrees of freedom.

An alternative embodiment of the invention provides that the base 15 on which the patient 13 rests is provided with an adjustable configuration. This adjustability is achieved in such a way that a hexapode is provided with which the base 15 can be adjusted. The provision of a hexapode for changing the position of the base 15 offers the advantage that the hexapode ensures an adjustability in six degrees of freedom. The base 15 and thus the patient 13 can be brought into any position in a continuous manner and with low need for space. Moreover, a hexapode ensures a highly precise and rapid adjustability of the base 15.

1. Radiotherapy system, consisting of at least one base on which a patient rests and of a radiation apparatus, especially a linear accelerator producing a treatment beam, characterized in that the direction of the treatment beam (12) is adjustable by means of at least one hexapode (3, 4, 5, 6, 7, 8, 9, 10).

2. A radiotherapy system according to claim 1, characterized in that the hexapode (3, 4, 5, 6, 7, 8, 9, 10) is arranged between linear accelerator (1) and a collimator (11).

3. A radiotherapy system according to claim 1 or 2, characterized in that platforms (3, 10) of the hexapode are configured in such a way that pass-through openings (16, 17) for allowing the passage of the treatment beam (12) are provided, especially that the platforms (3, 10) are provided with a ring-like arrangement.

4. A radiotherapy system according to one of the preceding claims, characterized in that the hexapode (3, 4, 5, 6, 7, 8, 9, 10) is controllable in such a way that the treatment beam (12) can be guided according to the shape of a tumor.

5. A radiotherapy system according to one of the preceding claims, characterized in that at least one sensor (20, 21) is provided on the hexapode (3, 4, 5, 6, 7, 8, 9, 10) and/or linear accelerator (1) and/or head (2) of the linear accelerator (1) and/or collimator (11) with which the position of the patient (13) on the base (15) can be detected.

6. A radiotherapy system according to claim 5, characterized in that two sensors (20, 21) are provided for detecting the position.

7. A radiotherapy system according to claim 5 or 6, characterized in that a control unit (30) is provided which by means of the sensors (20, 21) compares an actual position with a predetermined desired position of the patient (13) and controls hexapode (3, 4, 5, 6, 7, 8, 9, 10) in such a way that the treatment beam (12) meets an isocenter (14) in the patient at least approximately precisely.

8. A radiotherapy system according to one of the preceding claims, characterized in that the base (15) is adjustable by means of at least one hexapode.
